(19) United States
(12) Patent Application Publication
(10) Pub. No.: US 2016/0017275 A1
(43) Pub. Date: Jan. 21, 2016

(54) USE OF ATTENUATED PARASITE STRAINS FOR THE PREVENTION AND/OR TREATMENT OF EYE WOUNDS ASSOCIATED WITH AN INFECTION BY TOXOPLASMA GONDII

(71) Applicants: VITAMFERO, Tours (FR); UNIVERSITE FRANCOIS RABELAIS DE TOURS, Tours Cedex (FR)

(72) Inventors: Isabelle DIMIER-POISSON, TOURS (FR); Gildas LEMEE, TOURS (FR); Solen MORISSE, TOURS (FR); Edouard SECHE, ORLEANS (FR); Naoual TARFAOUI, TOURS (FR); Emre YURDUSEV, ANKARA (TR)

(21) Appl. No.: 14/773,560

(22) PCT Filed: Mar. 5, 2014
(86) PCT No.: PCT/FR2014/050505

§ 371 Date: Sep. 8, 2015

(30) Foreign Application Priority Data
Mar. 6, 2013 (FR) 13/51999

(51) Int. Cl. C12N 1/10; A61K 35/68; A61K 39/002; C12R 1/90

(52) U.S. Cl. CPC C12N 1/10; C12R 1/90; A61K 35/68; A61K 39/002; A61K 2039/522

(57) ABSTRACT

Strains of Toxoplasma gondii isolated from their natural environment for their use in the prevention and/or the treatment, in a mammal, of ocular lesions associated with an infection by an apicomplexan of the Sarcocystidae family.

The present invention relates to the use of attenuated living parasite strains for the prevention and/or the treatment of ocular lesions associated with a Toxoplasma gondii infection, in mammals and humans in particular.

The apicomplexans are obligate intracellular protozoan parasites having a life cycle which can involve several hosts. The phylum of these parasites subdivides into several families.

Toxoplasma gondii (T. gondii) belongs to the Sarcocystidae family. This protozoan exists in three infectious forms which vary depending on the host and the infectious stage:
the tachyzoite: proliferative and infectious form which multiplies asexually in the cells of intermediate hosts (i.e. all homeotherms) and definitive hosts (i.e. the felids and the cat in particular),
the bradyzoite: slow cell division form with a low metabolic rate of the parasite contained in cysts,
the sporozoite: a form contained in the oocysts, which results from the sexual multiplication of the parasite in the intestine of the definitive hosts (i.e. cat and other felids).

The cat, the definitive host of the parasite, becomes infected by ingesting parasitized prey containing cysts (or tachyzoites if the prey is in the acute phase of toxoplasmosis (Dubey, 2002, J. Parasitol., 88: 713-717)), or by the ingestion of oocysts. Infection in the cat leads to the formation, in its intestine, of gametocytes the fusion of which leads to the formation of oocysts which are then disseminated in the environment, via the faeces. These oocysts, which contain the sporozoites, sporulate and remain infectious for a very long time in the external environment. Their pathogenic character persists for at least one year.

After ingestion of oocysts by the definitive and intermediate hosts, the sporozoites released infect the enterocytes of the host and transform into tachyzoites which are disseminated in the organism. Under pressure from the immune system, the tachyzoite transforms into a bradyzoite which persists in cysts with a preferential tropism for the central nervous system, the retina and the muscles.

The ingestion of encysted tissues is the second cause of contamination of the definitive and intermediate hosts. After ingestion of cysts, the bradyzoites are released and infect the entero-epithelial cells and are transformed into tachyzoites, which are disseminated in the organism of the host and which, under pressure from the immune system, will again form intracystic bradyzoites.

The strains of T. gondii are classified into three types (Howe et al, 1995, J. Infect. Dis., 171: 1561-1566) depending on their degree of virulence in vivo: the strains of Type I (i.e. the RH strain) are highly virulent (Sibley et al, 1992, Nature, 359: 82-5) while the strains of Types II (i.e. the ME49, 76K or Prugniaud strains) and III (i.e. the CEP or M7741 strains) are relatively less virulent and generally establish chronic infections (Howe et al., 1997, J. Clin. Microbiol., 35: 1411-4). Moreover, numerous atypical strains that cannot be linked to the three first types are identified in particular in Africa and South America (Darde, 2008, Parasite, 15: 366-71; Rajendran et al, 2011, Infect. Genet. Evol., 12: 359-68; Mercier et al, 2010, PLoS NegL Trop. Dis., 4: e876, Ferreira et al., 2006, Infect. Genet. Evol., 6: 22-31).

Toxoplasma gondii is at the origin of an infectious disease: toxoplasmosis. Extremely widespread, with more than one third of the world population infected, the consequences of toxoplasmosis can be dramatic in two specific cases: (i) in pregnant women who are seronegative as the parasite can cross the placental barrier and infect the fœtus thus inducing a miscarriage or severe malformations or serious psychomotor disorders in the newborn and (ii) in immunosuppressed persons such as patients infected with the HIV virus in which toxoplasmosis, an opportunistic parasitosis, can lead to serious cerebral or cardiac disorders which are lethal if untreated.

In immunocompetent persons, toxoplasmosis is generally benign. However, for a few years, numerous publications have described the impact of this infectious disease on the eye. The retina constitutes a preferential site for encystment of the parasite. Thus, toxoplasmosis is the most frequent cause of intraocular inflammations and posterior uveitis in immunocompetent individuals. These intraocular inflammations can lead to a modification of the constituents of the ocular fluids (aqueous humour and vitreous humour) and thus impair visual function. Toxoplasmosis is also responsible for retinochoroiditis most often linked to the reactivation of parasites contained in the retinal cysts, which can result:
either from a congenital infection (i.e contracted in utero during pregnancy). The infection of the fœtus in the first weeks of pregnancy leads to major ocular lesions (microphthalmia, cataract, large nidi of retinochoroiditis). Ophthalmological monitoring of children infected with T. gondii in utero must be regularly carried out during the first seven years of the life of the child. The functional signs which are monitored (i.e. visual impairment, floaters, scotoma) must immediately alert the practitioner to a reactivation of T. gondii characterized by a multiplying phase of the parasite.
or from an acquired infection. The congenital infection does not constitute the only etiology of ocular toxoplasmosis. In fact, the role of the infections of acquired origin has been demonstrated in numerous cases of ocular toxoplasmosis, in particular in several members of one and the same sibling group or when the prevalence is high in certain geographical areas. During ocular toxoplasmoses of acquired origin, the ocular manifestations can be concomitant with the primary infection or deferred, sometimes for years or decades afterwards.

The reactivation rate of T. gondii resulting from an infection of acquired origin is comparable to that resulting from a congenital infection and whatever their etiological origin, the lesions generated on the retina can lead to a deterioration or a loss of vision if they are located close to the macula or the optic nerve.

The prevalence and the incidence of ocular toxoplasmosis is highly dependent on the geographical area. In France, where, as in Europe, Type II strains predominate, the prevalence of ocular toxoplasmosis in the population has not been assessed with certainty but the incidence of the disease is assumed to be 2% of the patients infected and would therefore involve approximately 800,000 patients. In certain regions of the world, and in particular in South America, where Type I strains of T. gondii predominate (i.e. the more virulent), the incidence of ocular toxoplasmosis is markedly higher. Thus, in the region of Erechim, a town situated in the south of Brazil, toxoplasmic retinochoroidal scars have been found in 17.7% of the subjects examined out of a total population of 2 million inhabitants, 20,000 people have lost the use of one eye and 5,000 people are completely blind (Jones et al, 2006, Emerg Infect Dis, 12: 582-587; Silveira et al., 2009, Expert Rev. Anti infect. Ther. 7: 905-908).

Diagnosis of ocular toxoplasmosis is essentially clinical. When the lesions observed in the fundus of the eye are atypical, serological analysis of the anti-T. gondii antibodies can be carried out, not only in the serum of the patients, but also in the aqueous humour of the eye. Measurements of the levels of anti-T. gondii IgG and of the levels of total IgG in these two chambers make it possible to determine the value of the Desmonts coefficient. This coefficient corresponds to the anti-T. gondii IgG/total IgG ratio in the aqueous humour over the anti-T. gondii IgG/total IgG ratio in the blood. When this ratio is greater than 3, it is considered that there is a local synthesis of anti-T. gondii antibodies indicating an intraocular infection. When this ratio is less than 2, the local production of anti-T. gondii antibodies is not demonstrated, although an ocular toxoplasmosis cannot be ruled out. A value comprised between 2 and 3 is doubtful for confirming a local production of anti-T. gondii antibodies. In the last two cases, the presence of the parasite in the aqueous humour can be detected by amplification of the DNA of T. gondii using the Polymerase Chain Reaction technique.

Current treatments (Engstrom et al, 1991, Am. J. Ophthalmol., 111: 601-610) are based on the use of pyrimethamine or sulfadiazine. These antiparasitics or antibiotics, by inhibiting the metabolism of folic acid, block the synthesis of the nucleic acids of the parasite. These compounds are active on the tachyzoites, but are inactive on the bradyzoites. Cotrimoxazole and clindamycin also act on the tachyzoites. Azithromycin has an action in vitro on the bradyzoites which proves to be weak in vivo on this parasitic form. Atovaquone has the best action potential in vivo, both on the tachyzoites and on the bradyzoites. These compounds must be used at high concentration in order to cross the blood-ocular barrier and reach sufficient levels at the retina. For this reason, these medicinal products can have dramatic side effects (Lyell's syndrome, agranulocytosis, pseudo-membraneous colitis) and require a supplement of folates for the patient. The use of these compounds can be associated with corticotherapy. The aim of the corticoids is to limit the inflammatory reaction associated with retinochoroiditis of toxoplasmic origin. Prednisone constitutes the reference corticoid. The administration of corticoids is however reserved for immunocompetent patients.

As these compounds do not have a cysticidal effect, they cannot therefore prevent the reactivation of encysted T. gondii leading to recurrences of the disease, estimated at 15% at two years. As a result, they are only prescribed when the visual function is threatened (macular or papillary damage). The aim of the treatment is then to activate healing of the existing lesions. The therapeutic treatments make it possible to limit the damage during a reactivation of the encysted parasite but do not in any event prevent subsequent reactivations.

Ocular toxoplasmosis which for a long time has been under-estimated, therefore has serious consequences and this infection is now part of health screening programmes set up in France, Brazil, etc. The marketing of an effective anti-toxoplasmosis vaccine, subsequently limiting the spread of the parasite in the ocular tissues and the clinical consequences of subsequent outbreaks, is a worthwhile and promising strategy for limiting the impact of this infectious disease (Silveira et al., 2009, Expert Rev. Anti infect. Ther. 7: 905-908).

As a result a real need exists to develop an agent for the prevention and/or treatment of ocular lesions in a mammal, in particular a mammal already infected with T. gondii or a mammal susceptible to being infected with T. gondii and developing a toxoplasmosis.

Recently, an attenuated living vaccinal strain of T. gondii has been developed by knockout of two genes coding for the TgMIC1 and TgMIC3 proteins (EP 1 703 914 B1 and U.S. Pat. No. 7,964,185 B2/Cerede et al, 2005 J. Exp. Med., 201: 453-63). This strain, called Toxo mic1-3 KO, generates a strong and specific immune response against T. gondii and makes it possible to prevent the effects of a subsequent infection in mice (Ismaël et al, 2005, J. Infect. Dis., 194: 1176-1183) and also ewes (Mevelec et al, 2010, Vet. Res., 41: 49-60). It has also been demonstrated that the virulence in vivo is only slightly affected by the inactivation of TgMIC1 or of TgMIC3 in isolation; on the other hand, it is greatly reduced by the simultaneous inactivation of both proteins, demonstrating the synergistic role of the two proteins (Cerede et al, 2005 J. Exp. Med., 201: 453-63).

One of the purposes of the invention is to provide an agent making it possible to reduce, in a mammal, the inflammation in the eye caused by a T. gondii infection.

Another purpose of the invention is to reduce the inflammatory reaction produced in the eye at the time of the reactivation of T. gondii in a mammal already infected with T. gondii.

Another purpose of the invention is to provide an agent making it possible to reduce the number of cysts, in particular intraretinal cysts, thus enabling the prevention of ocular lesions linked to the reactivation of T. gondii.

Yet another purpose of the invention is to provide a vaccine against the ocular lesions generated by T. gondii.

A subject of the present invention is strains of Toxoplasma gondii isolated from their natural environment for their use in the prevention and/or the treatment, in a mammal, of the ocular lesions associated with an infection by an apicomplexan of the Sarcocystidae family.

A subject of the present invention is strains of Toxoplasma gondii isolated from their natural environment for their use in the prevention and/or the treatment, in a mammal, of the ocular lesions associated with an infection by an apicomplexan of the Sarcocystidae family, said strains having an attenuated virulence in comparison with a virulent strain of T. gondii of RH type.

A subject of the present invention is strains of Toxoplasma gondii isolated from their natural environment for their use in the prevention or the treatment, in a mammal, of the ocular lesions associated with an infection with by an apicomplexan of the Sarcocystidae family, said strains having an attenuated virulence in comparison with a virulent strain, i.e. a strain having a virulence substantially identical to the virulence of the strain from which the strain with attenuated virulence has been obtained.

By “prevention”, is meant the prophylaxis having the aim of preventing the onset or the propagation of a disease. This involves in particular protecting an individual predisposed to contracting and developing ocular lesions associated with an infection by an apicomplexan of the Sarcocystidae family. It involves in particular protecting a mammal exposed to the risk of contamination via its environment.

By “treatment”, is meant not only the inhibition of the progression of the pathology, but also the attenuation, even the disappearance, of the symptoms linked to this pathology. The treatment has the aim of reducing the extent of the symptoms until they completely disappear, allowing the individual to return to a normal physiological state.

By “mammal”, is meant human beings, pets, and commercial or farm animals, which are of economic and commercial interest to the agricultural and food industries.

By “strains of Toxoplasma gondii”, is meant the strains of Toxoplasma gondii which have an attenuated virulence, less than the virulence of the strains of T. gondii of RH type from which they derive, but which nevertheless retain an immunogenicity identical to that of the strains of T. gondii of RH type in order to be able use them in the prevention or the treatment of a pathology associated with an infection by an apicomplexan of the Sarcocystidae family. This attenuated virulence can result from the knockout of at least one gene linked to the virulence of the parasite. This gene knockout can take place during a natural process of evolution of the species or be carried out in vitro by the molecular biology techniques well known to a person skilled in the art. In the first case, the gene knockout from the genome takes place randomly, while in the other case, the gene knockout is targeted on one or more specific gene(s). Whether of natural origin or resulting from human intervention, this gene knockout leads to the absence of expression of the protein encoded by the knocked-out gene(s) or to the expression of one or more non-functional proteins. The in vitro modification of the gene pool of Toxoplasma gondii gives to the strain a mutant character, as opposed to the wild strain from which it derives. The wild-type strains of parasites not only have an immunogenic potential but are also virulent, i.e. they are capable of inducing a pathology associated with Toxoplasma gondii (i.e. toxoplasmosis), making their use unsuitable in the context of the present invention.

By “pathologies associated with an infection with an apicomplexan of the Sarcocystidae family”, is meant the diseases resulting from an infection with a protozoan belonging to the Apicomplexa phylum, and in particular the parasites belonging to the sarcocystidae family which contains the genus Toxoplasma.

According to a particular embodiment, in the use according to the present invention of the strains of Toxoplasma gondii isolated from their natural environment, said mammal is a human being or an animal.

According to another embodiment, in the use according to the present invention of the strains of Toxoplasma gondii isolated from their natural environment, said strains of Toxoplasma gondii have at least one adhesin MIC-1 and/or one adhesin MIC-3 inactivated by a genetic modification involving at least one of the mic-1 and/or mic-3 genes.

According to another particular embodiment, in the use according to the present invention of the strains of Toxoplasma gondii isolated from their natural environment, said strains of Toxoplasma gondii have at least one adhesin MIC-1 and/or one adhesin MIC-3 inactivated by the deletion of at least one of the mic-1 and/or mic-3 genes.

By “an adhesin MIC-1 and/or an adhesin MIC-3”, is meant the proteins of the micronemes, also called adhesins, MIC-1 and/or MIC-3 which play a role in the mobility, migration or cell invasion by the parasites of the Apicomplexa phylum in their host. These proteins have binding modules which allow them to bind to the cells of the host.

By “an inactivated adhesin”, is meant an adhesin the function of which can no longer be carried out within the cell. An adhesin is inactivated when it is not produced or when it is produced but does not have functional activity. The inactivation can also be the consequence of ineffective or inadequate post-translational modifications (i.e. glycosylation, isoprenylation, phosphorylation, sulphation, amidation, acetylation, alkylation) of the adhesin which do not allow it to carry out its function. The inactivation of an adhesin can also be obtained indirectly by altering or suppressing the expression of one or more other proteins (in particular other adhesins) which bind to the adhesin in order to form a functional complex. The destructuring of such a complex leads to a loss of function of the adhesin.

By “genetic modification”, is meant any mutation carried out in the nucleotide sequence of a gene leading to the absence of expression of the protein encoded by this gene or leading to the expression of a non-functional form of the protein encoded by this gene. This operation requires the intervention of a person skilled in the art when it is carried out in vitro. This mutation can consist of the deletion of all or part of the gene, or of its coding region, or of its promoter region, or the insertion or substitution of nucleotides in the nucleotide sequence of the gene.

By “mic-1 gene”, is meant the gene coding for the MIC-1 microneme protein, also called adhesin MIC-1. This protein contains several modules the binding domains of which specifically bind lactose. The MIC-1 protein is also capable of binding to the surface of the host cells.

By “mic-3 gene”, is meant the gene coding for the MIC-3 microneme protein, also called adhesin MIC-3. This protein homodimerizes in order to form a complex of 90 kDa. MIC-3 comprises domains of EGF type and a domain of the lectin type. The MIC-3 protein is also capable of binding to the surface of the host cells.

According to yet another embodiment, in the use according to the present invention of the strains of Toxoplasma gondii isolated from their natural environment, said strains of Toxoplasma gondii have the two adhesins MIC-1 and MIC-3 inactivated by a genetic modification involving the two mic-1 and mic-3 genes.

According to yet another particular embodiment, in the use according to the present invention of the strains of Toxoplasma gondii isolated from their natural environment, said strains of Toxoplasma gondii have the two adhesins MIC-1 and MIC-3 inactivated by the deletion of the two mic-1 and mic-3 genes.

By “genetic modification involving the two mic-1 and mic-3 genes”, is meant the mutation carried out in the nucleotide sequence of the mic-1 gene and in that of the mic-3 gene. This double mutation leads to the absence of expression of the MIC-1 and MIC-3 proteins or leads to the expression of a non-functional form of the MIC-1 and MIC-3 proteins. This mutant strain of Toxoplasma gondii is called Toxo mic1-3 KO and has a very attenuated virulence in comparison with the wild-type strains of T. gondii of RH type from which it derives. The Toxo mic1-3 KO strains however retain a strong immunogenicity. The detailed construction of the Toxo mic1-3 KO strain is described in the documents Cerede et al, 2005 J. Exp. Med., 201: 453-63, U.S. Pat. No. 7,964,185 B2 and EP 1 703 914 B1.

The Toxo mic1-3 KO strains have retained their ability to colonize the target tissues without the development of any pathogenic phenomenon caused by the administration of said strains to a mammal. The knockout of the mic1 and mic3 genes in no way alters the immunogenic potential of these strains, but considerably reduces their virulence in comparison with a virulent strain, i.e. a strain with a virulence substantially identical to the virulence of the strain from which the strain with attenuated virulence has been obtained.

According to another embodiment, in the use according to the present invention of the strains of Toxoplasma gondii isolated from their natural environment, said apicomplexan of the Sarcocystidae family is Toxoplasma gondii.

By “Toxoplasma gondii”, is meant the protozoans of the Apicomplexa phylum capable of causing congenital malformations or miscarriages in most warm-blooded mammals and birds. These parasites are in particular capable of causing toxoplasmosis in humans, pigs, ewes, rodents or any mammal whether it has a mature or deficient immune system.

According to another embodiment, in the use according to the present invention of the strains of Toxoplasma gondii isolated from their natural environment, said ocular lesions belong to the group comprising or constituted by intraocular inflammations, uveitis, hyalitis or retinochoroiditis.

By “intraocular inflammations”, is meant any secretion of cytokines and chemokines participating in the immune response and any recruitment or activation of the cells of the immune systems contained in the eye.

By “uveitis”, is meant inflammation of the uvea, a vascularized complex making it possible to nourish the eye and which comprises the iris, the ciliary body (anatomical element to which the ligaments retaining the crystalline lens are connected) and the choroid. This inflammation can be caused by a virus, a bacterium or a parasite or be due to an auto-immune disease. Uveitis can be anterior, intermediate or posterior depending on the compartments of the eye that it affects. Uveitis can be painful or not and be associated with a decline in visual acuity. The eye can appear red with, in particular, lacrimation or photophobia.

By “hyalitis”, is meant inflammation of the vitreous body of the eye which corresponds to intermediate uveitis.

By “retinochoroiditis”, is meant clinical entities which comprise any lesion of the retina-choroid complex. Always inflammatory, they can be of infectious or auto-immune origin. Retinochoroiditis therefore corresponds to an inflammation of the posterior uvea, i.e. the retina and the choroid.

According to a more particular embodiment, in the use according to the present invention of the strains of Toxoplasma gondii isolated from their natural environment, said ocular lesions are caused by a primary infection of said mammal with T. gondii.

By “primary infection”, is meant the first contact of said mammal with the parasite T. gondii. This primary infection leads to an immune response characterized in particular by the production of anti-T. gondii antibodies and by the activation of a cellular immune response directed specifically against T. gondii.

According to a yet more particular embodiment, in the use according to the present invention of the strains of Toxoplasma gondii isolated from their natural environment, said ocular lesions are caused by a reactivation of the asexual form of T. gondii.

By “reactivation of the asexual form of T. gondii”, is meant the conversion of the bradyzoites of T. gondii to tachyzoites and the release of the latter after rupture of the cyst, thus inducing contamination of the cells. The reactivation of T. gondii can be caused by a deficiency in the immune system of the infected mammal.

According to a yet more particular embodiment, in the use according to the present invention of the strains of Toxoplasma gondii isolated from their natural environment, said ocular lesions are caused by a primary infection of said mammal by T. gondii and by a reactivation of the asexual form of T. gondii.

According to a particular embodiment, in the use according to the present invention of strains of Toxoplasma gondii isolated from their natural environment, said strains are placed in contact with said mammal at a rate of 100 to 10⁸ tachyzoites.

By “tachyzoite”, is meant the rapidly replicating and asexual form of Toxoplasma gondii. The tachyzoite has a size of 5-8×2-3 μm. The apical part of the parasite comprises conoids which participate in the penetration of the parasite into the host cell. The micronemes, the rhoptries and the dense granules constitute the three major organelles of the tachyzoite which also comprises a nucleus, an apicoplast, a Golgi apparatus, an endoplasmic reticulum and an organelle close to the mitochondrion.

The effective dose of tachyzoites for the prophylactic treatment of mammals makes it possible to limit the infection or the transmission of the pathogenic agent responsible for toxoplasmosis. Another purpose is to prevent the occurrence of new intraocular lesions during reactivations of the encysted parasite. Such a treatment can be adapted and/or repeated as many times as necessary by a person skilled in the art depending on the age and immunological status of the mammal.

Placing the tachyzoites in contact with the mammal can be carried out not only on a mammal presenting the symptoms inherent to toxoplasmosis, but also on a mammal presenting none of these symptoms but which presents a risk of infection by virulent strains of Toxoplasma gondii capable of inducing toxoplasmosis and therefore these symptoms.

According to a particular embodiment, the strains of Toxoplasma gondii isolated from their natural environment for their use according to the present invention are in a galenic form selected from the group comprising or constituted by liquid suspensions, solid or liquid dispersions, powders, pastes or lyophilizates.

The galenic form is adapted by a person skilled in the art depending on the chosen administration method. All the standard administration methods can be envisaged: by enteral route (per os for example) by parenteral route (intravenous, intramuscular or intraperitoneal injection for example) or by intranasal spray.

According to a more particular embodiment, the strains of Toxoplasma gondii isolated from their natural environment for their use according to the present invention can be combined with at least one other antigen, at least one adjuvant, at least one stabilizer, at least one preservative, at least one vector or a mixture of these products making it possible to stimulate and increase the immune response of said mammal.

By “antigen”, is meant any natural or recombinant protein, in its native or mutated form, originating from a parasite or from a pathogenic agent other than Toxoplasma gondii capable of inducing a cellular or humoral immune response in a mammal. The purpose of the combination of the mutant strain of Toxoplasma gondii with such an antigen is to amplify the immune response of the mammal and thus give it better protection vis-à-vis an apicomplexan infection.

By “adjuvant”, is meant any substance capable of reinforcing and prolonging the immune response directed against the target antigen. The mechanism involved in order to render the immune response more effective is dependent on the adjuvant used. The adjuvants are substances well known to a person skilled in the art which include in particular aluminium salts, squalene, saponins, bacterial constituents or toxins, or also certain proteins (peptone, albumin, casein).

By “stabilizer or preservatives”, is meant the compounds allowing the perfect preservation of strains of Toxoplasma gondii in their packaging. The purpose of these compounds is to guarantee the viability of the strains of T. gondii. The stabilizers or preservatives are substances well known to a person skilled in the art which include in particular carbon hydrates (sorbitol, mannitol, lactose, sucrose, glucose, dextran, trehalose), polar organic solvents such as DMSO (dimethylsulphoxide), polysorbates.

By “vector” is meant an entity used to make a gene of interest penetrate into a cell. The vectors are substances well known to a person skilled in the art which include in particular nanoparticles, ISCOMs (i.e. “immune stimulatory complexes”), etc.

By “increase the immune response”, is meant the activation of the different pathways of the immune system of the mammal by the combination constituted by the mutant strain of T. gondii with a second antigen. The innate immune response is activated by increasing the synthesis of cytokines of distinct categories such as the interferons or the interleukins. Interleukin-12 (IL-12) is capable of stimulating the Natural Killer (NK) cells. The cells thus stimulated will secrete interferon-γ (IFN-γ) which plays a major role in protection against the intracellular parasites such as T. gondii. In an adult mammal having a mature and competent immune system, the immune response is based on an adaptive immunity (specific proliferation in response to foreign antigens by the T lymphocytes CD4+ and CD8+ in addition to the innate response.

According to another particular embodiment, the strains of Toxoplasma gondii isolated from their natural environment for their use according to the present invention can be combined with at least one antiparasitic compound or an antibiotic selected from the group comprising or constituted by pyrimethamine, sulphadiazine, cotrimoxazole, clindamycin, azithromycin or atovaquone.

According to another more particular embodiment, the strains of Toxoplasma gondii isolated from their natural environment for their use according to the present invention can be combined with at least one antiparasitic compound or an antibiotic selected from the group comprising or constituted by pyrimethamine, sulphadiazine, cotrimoxazole, clindamycin, azithromycin or atovaquone and with a corticoid.

The present invention also relates to a method for preventing the occurrence of, and/or for treating, in a mammal, ocular lesions caused by one or more apicomplexans of the Sarcocystidae family comprising a step of administration of tachyzoites of one or more mutant strains of Toxoplasma gondii with attenuated virulence to said mammal making it possible to reduce the number of ocular lesions.

According to a particular embodiment, in the method according to the present invention, the mutant strains of Toxoplasma gondii have at least one adhesin MIC-1 or one adhesin MIC-3 inactivated by a genetic modification involving at least one of the mic-1 or mic-3 genes.

According to a more particular embodiment, in the method according to the present invention, the mutant strains of Toxoplasma gondii have at least one adhesin MIC-1 and/or one adhesin MIC-3 inactivated by the deletion of at least one of the mic-1 and/or mic-3 genes.

According to another particular embodiment, in the method according to the present invention, the mutant strains of Toxoplasma gondii have the two adhesins MIC-1 and MIC-3 inactivated by a genetic modification involving the two mic-1 and mic-3 genes.

According to another more particular embodiment, in the method according to the present invention, the mutant strains of Toxoplasma gondii have the two adhesins MIC-1 and MIC-3 inactivated by the deletion of the two mic-1 and mic-3 genes.

According to another particular embodiment, in the method according to the present invention, the mammal is a human being or an animal.

In a non-vaccinated mammal according to the method of the present invention, a challenge infection leads to the high intraocular synthesis of IFN-γ. The method according to the present invention inhibits this intraocular synthesis of IFN-γ resulting from the challenge infection.

The present invention also relates to a method for preventing the occurrence and/or treating, in a mammal, of intraocular inflammation caused by one or more apicomplexans of the Sarcocystidae family comprising a step of administration of tachyzoites of one or more mutant strains of Toxoplasma gondii with attenuated virulence to said mammal.

According to another more particular embodiment, in the method according to the present invention, the administration of tachyzoites of one or more mutant strains of Toxoplasma gondii is carried out by enteral route or by parenteral route.

The following figures and examples are given only by way of illustration of the subject-matter of the present invention of which they in no way constitute a limitation.

### DESCRIPTION OF THE FIGURES

FIG. 1A: ophthalmological clinical signs in Swiss Webster (OF1) mice 4 weeks after the challenge infection.

The determination of the ophthalmological clinical signs is carried out under binocular loupe on each eye of each of the mice 4 weeks after the mice have been infected by oral route with 50 cysts of the 76K strain of Toxoplasma gondii. The mice were vaccinated with 100 tachyzoites of the Toxo mic1-3 KO strain 4 weeks before the challenge infection (black column) or were not vaccinated (grey column).

on the x-axis:
(a) ocular lesions of the uveitis/hyalitis type,
(b) ocular lesions of the haemorrhagic type,
(c) ocular lesions of the corneal opacity type,
(d) ocular lesions of the cataract type.

on the y-axis: percentage of eyes presenting lesions.

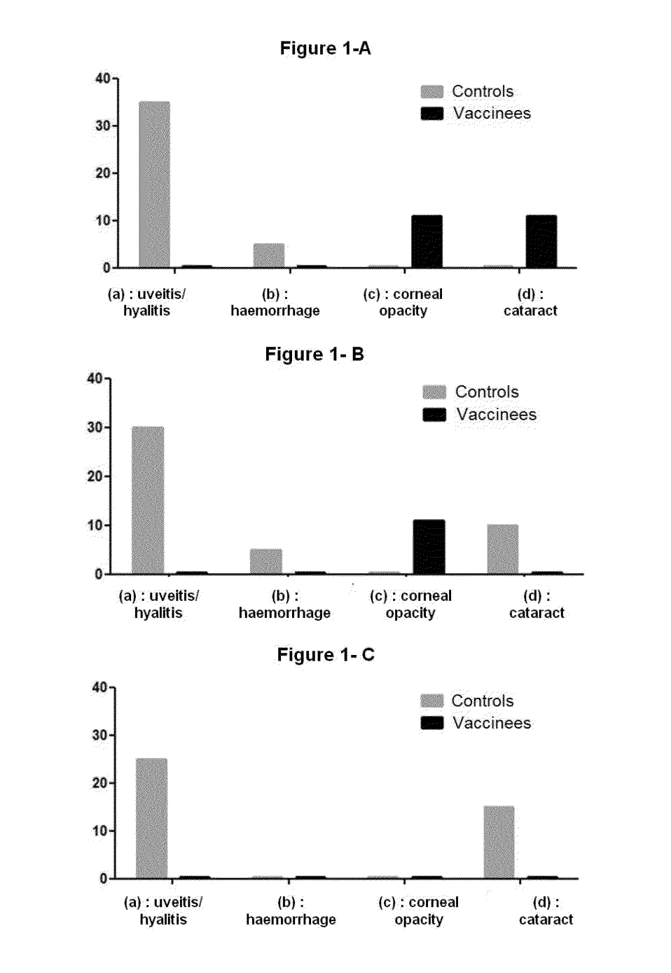

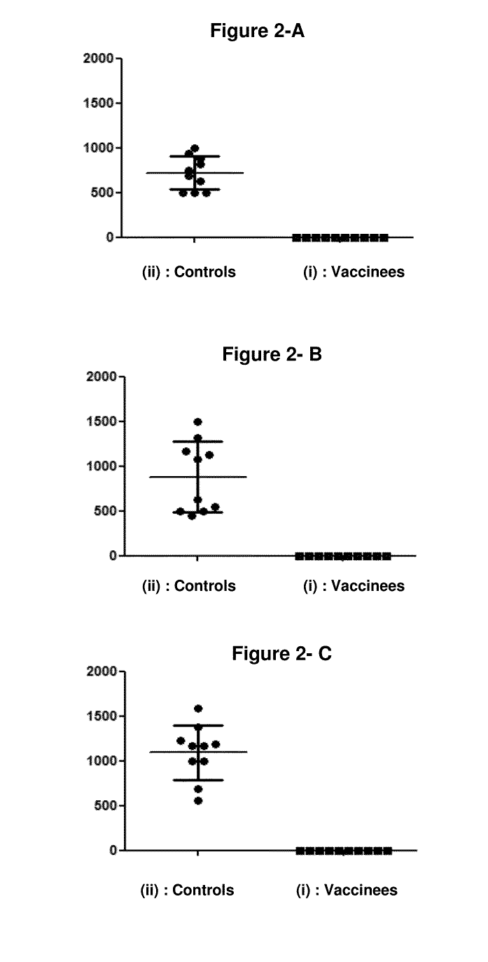

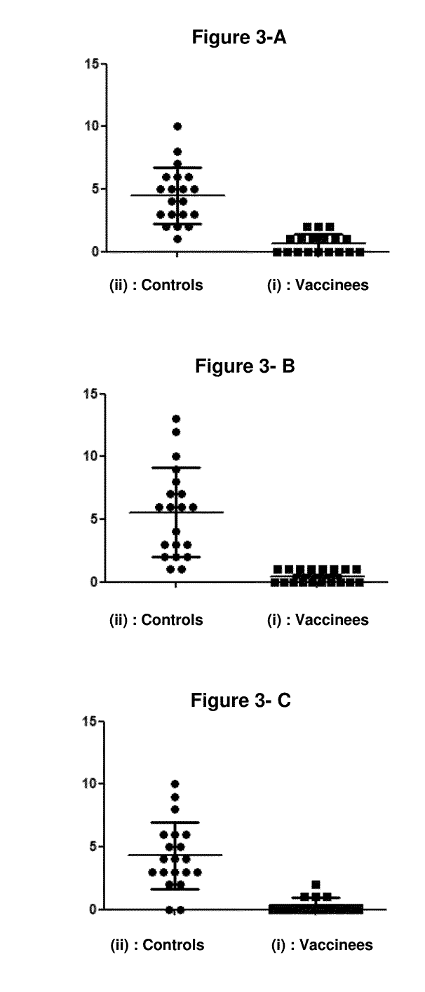

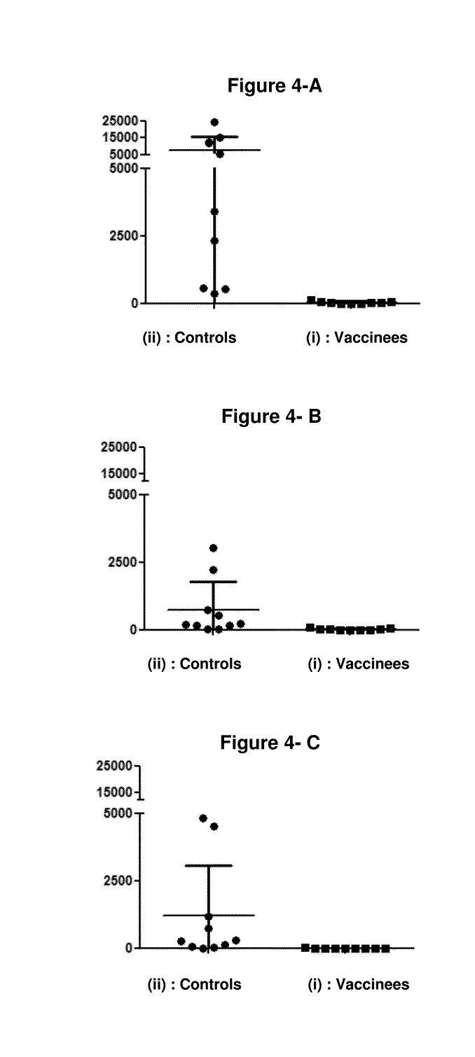

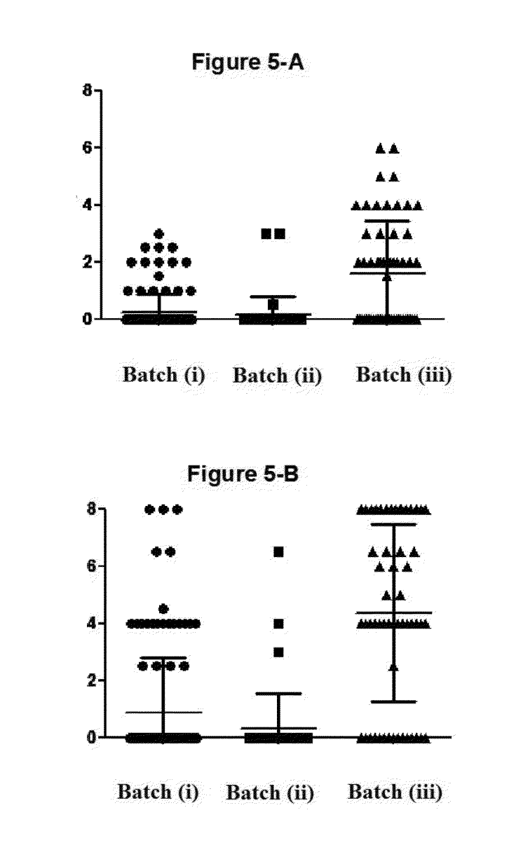

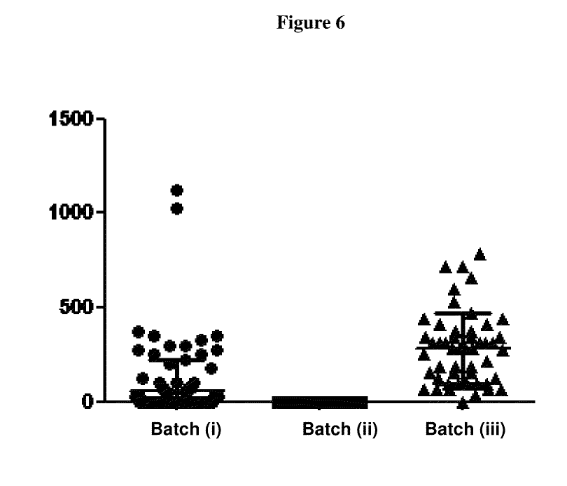

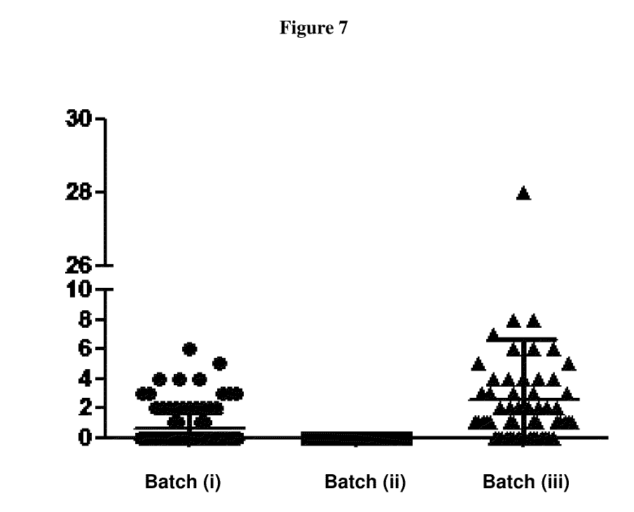

FIG. 1B: ophthalmological clinical signs in Swiss Webster (OF1) mice 8 weeks after the challenge infection.

The determination of the ophthalmological clinical signs is carried out under binocular loupe on each eye of each of the mice 8 weeks after the mice have been infected by oral route with 50 cysts of the 76K strain of Toxoplasma gondii. The mice were vaccinated with 100 tachyzoites of the Toxo mic1-3 KO strain 4 weeks before the challenge infection (black column) or were not vaccinated (grey column).

on the x-axis:
(a) ocular lesions of the uveitis/hyalitis type,
(b) ocular lesions of the haemorrhagic type,
(c) ocular lesions of the corneal opacity type,
(d) ocular lesions of the cataract type.

on the y-axis: percentage of eyes presenting lesions.

FIG. 1C: ophthalmological clinical signs in Swiss Webster (OF1) mice 12 weeks after the challenge infection.

The determination of the ophthalmological clinical signs is carried out under binocular loupe on each eye of each of the mice 12 weeks after the mice have been infected by oral route with 50 cysts of the 76K strain of Toxoplasma gondii. The mice were vaccinated with 100 tachyzoites of the Toxo mic1-3 KO strain 4 weeks before the challenge infection (black column) or were not vaccinated (grey column).

on the x-axis:
(a) ocular lesions of the uveitis/hyalitis type,
(b) ocular lesions of the haemorrhagic type,
(c) ocular lesions of the corneal opacity type,
(d) ocular lesions of the cataract type.

on the y-axis: percentage of eyes presenting lesions.

FIG. 2A: evaluation of the number of intracerebral cysts in Swiss Webster (OF1) mice 4 weeks after the challenge infection.

Counting the number of intracerebral cysts was carried out on the basis of a homogenate of mouse brain, 4 weeks after the mice had been infected by oral route with 50 cysts of the 76K strain of Toxoplasma gondii. Each dot represents a mouse. Eight to ten counts are carried out on 10 μL of this homogenate using a binocular microscope in Malassez cells. The average is then calculated and applied to the entire initial volume in order to evaluate the number of intracerebral cysts.

on the x-axis: batches of Swiss Webster (OF1) mice:
batch (i): mice having received 100 tachyzoites of the Toxo mic1-3 KO strain by intraperitoneal route then infected with 50 cysts of the 76K strain of Toxoplasma gondii (black squares),
batch (ii): mice only infected with 50 cysts of the 76K strain of Toxoplasma gondii (black circles).

on the y-axis: number of intracerebral cysts.

FIG. 2B: evaluation of the number of intracerebral cysts in Swiss Webster (OF1) mice 8 weeks after the challenge infection.

Counting the number of intracerebral cysts was carried out on the basis of a homogenate of mouse brain, 8 weeks after the mice had been infected by oral route with 50 cysts of the 76K strain of Toxoplasma gondii. Each dot represents a mouse. Eight to ten counts are carried out on 10 μL of this homogenate using a binocular loupe microscope in Malassez cells. The average is then calculated and applied to the entire initial volume in order to evaluate the number of intracerebral cysts.

on the x-axis: batches of Swiss Webster (OF1) mice:
batch (i): mice having received 100 tachyzoites of the Toxo mic1-3 KO strain by intraperitoneal route then infected with 50 cysts of the 76K strain of Toxoplasma gondii (black squares),
batch (ii): mice only infected with 50 cysts of the 76K strain of Toxoplasma gondii (black circles).

on the y-axis: number of intracerebral cysts.

FIG. 2C: evaluation of the number of intracerebral cysts in Swiss Webster (OF1) mice 12 weeks after the challenge infection.

Counting the number of intracerebral cysts was carried out on the basis of a homogenate of mouse brain, 12 weeks after the mice had been infected by oral route with 50 cysts of the 76K strain of Toxoplasma gondii. Each dot represents a mouse. Eight to ten counts are carried out on 10 μL of this homogenate using a binocular loupe microscope in Malassez cells. The average is then calculated and applied to the entire initial volume in order to evaluate the number of intracerebral cysts.

on the x-axis: batches of Swiss Webster (OF1) mice:
batch (i): mice having received 100 tachyzoites of the Toxo mic1-3 KO strain by intraperitoneal route then infected with 50 cysts of the 76K strain of Toxoplasma gondii (black squares),
batch (ii): mice only infected with 50 cysts of the 76K strain of Toxoplasma gondii (black circles).

on the y-axis: number of intracerebral cysts.

FIG. 3A: evaluation of the number of intra-retinal cysts in Swiss Webster (OF1) mice 4 weeks after the challenge infection.

Counting the number of intra-retinal cysts was carried out on the retina-choroid complex of the enucleated eyes of the mice 4 weeks after the mice had been infected by oral route with 50 cysts of the 76K strain of Toxoplasma gondii. Each dot represents a mouse.

on the x-axis: batches of Swiss Webster (OF1) mice:
batch (i): mice having received 100 tachyzoites of the Toxo mic1-3 KO strain by intraperitoneal route then infected with 50 cysts of the 76K strain of Toxoplasma gondii (black squares),
batch (ii): mice only infected with 50 cysts of the 76K strain of Toxoplasma gondii (black circles).

on the y-axis: number of intra-retinal cysts.

FIG. 3B: evaluation of the number of intra-retinal cysts in Swiss Webster (OF1) mice 8 weeks after the challenge infection.

Counting the number of intra-retinal cysts was carried out on the retina-choroid complex of the enucleated eyes of the mice 8 weeks after the mice had been infected by oral route with 50 cysts of the 76K strain of Toxoplasma gondii. Each dot represents a mouse.

on the x-axis: batches of Swiss Webster (OF1) mice:
batch (i): mice having received 100 tachyzoites of the Toxo mic1-3 KO strain by intraperitoneal route then infected with 50 cysts of the 76K strain of Toxoplasma gondii (black squares),
batch (ii): mice only infected with 50 cysts of the 76K strain of Toxoplasma gondii (black circles).

on the y-axis: number of intra-retinal cysts.

FIG. 3C: evaluation of the number of intra-retinal cysts in Swiss Webster (OF1) mice 12 weeks after the challenge infection.

Counting the number of intra-retinal cysts was carried out on the retina-choroid complex of the enucleated eyes of the mice 12 weeks after the mice had been infected by oral route with 50 cysts of the 76K strain of Toxoplasma gondii. Each dot represents a mouse.

on the x-axis: batches of Swiss Webster (OF1) mice:
batch (i): mice having received 100 tachyzoites of the Toxo mic1-3 KO strain by intraperitoneal route then infected with 50 cysts of the 76K strain of Toxoplasma gondii (black squares),
batch (ii): mice only infected with 50 cysts of the 76K strain of Toxoplasma gondii (black circles).

on the y-axis: number of intra-retinal cysts.

FIG. 4A: assay of the IFN-γ in the aqueous humour of Swiss Webster (OF1) mice 4 weeks after the start of the challenge infection.

The assay of interferon gamma (IFN-γ) in the aqueous humour was carried out 4 weeks after the mice had been infected by oral route with 50 cysts of the 76K strain of Toxoplasma gondii. Each dot represents a mouse.

on the x-axis: batches of Swiss Webster (OF1) mice
batch (i): mice having received 100 tachyzoites of the Toxo mic1-3 KO strain by intraperitoneal route then infected with 50 cysts of the 76K strain of Toxoplasma gondii (black squares),
batch (ii): mice only infected with 50 cysts of the 76K strain of Toxoplasma gondii (black circles).

on the y-axis: concentration of IFN-γ in pg/mL.

FIG. 4B: assay of the IFN-γ in the aqueous humour of Swiss Webster (OF1) mice 8 weeks after the start of the challenge infection.

The assay of the interferon gamma (IFN-γ) in the aqueous humour was carried out 8 weeks after the mice had been infected by oral route with 50 cysts of the 76K strain of Toxoplasma gondii. Each dot represents a mouse.

on the x-axis: batches of Swiss Webster (OF1) mice
batch (i): mice having received 100 tachyzoites of the Toxo mic1-3 KO strain by intraperitoneal route then infected with 50 cysts of the 76K strain of Toxoplasma gondii (black squares),
batch (ii): mice only infected with 50 cysts of the 76K strain of Toxoplasma gondii (black circles).

on the y-axis: concentration of IFN-γ in pg/mL.

FIG. 4C: assay of the IFN-γ in the aqueous humour of Swiss Webster (OF1) mice 12 weeks after the start of the challenge infection.

The assay of the interferon gamma (IFN-γ) in the aqueous humour was carried out 12 weeks after the mice had been infected by oral route with 50 cysts of the 76K strain of Toxoplasma gondii. Each dot represents a mouse.

on the x-axis: batches of Swiss Webster (OF1) mice
batch (i): mice having received 100 tachyzoites of the Toxo mic1-3 KO strain by intraperitoneal route then infected with 50 cysts of the 76K strain of Toxoplasma gondii (black squares),
batch (ii): mice only infected with 50 cysts of the 76K strain of Toxoplasma gondii (black circles).

on the y-axis: concentration of IFN-γ in pg/mL.

FIG. 5A: ophthalmological clinical signs in Swiss Webster (OF1) baby mice aged 4 weeks and infected in utero with Toxoplasma gondii.

The determination of the ophthalmological clinical signs in baby mice aged 4 weeks and infected in utero with Toxoplasma gondii is carried out using a binocular loupe under general anaesthesia by inhalation of gaseous isoflurane at 2.5% (oxygen at 3 L/min). In order to evaluate the inflammation, the following scoring is used:
Stage 0: No inflammation,
Stage 1: Moderate Tyndall effect in the anterior or vitreous chamber,
Stage 2: Severe Tyndall effect in the anterior or vitreous chamber and/or dilatation of the blood vessels of the iris and/or of the conjunctiva/sclera,
Stage 3: Clouding of the cornea and retrocorneal precipitates and/or very severe hyalitis,
Stage 4: Secondary cataract.
   Each dot represents the cumulative clinical stage for a baby mouse (cumulative clinical stage being the sum of the clinical stages of the 2 eyes per baby mouse).

on the x-axis: batches of Swiss Webster (OF1) baby mice:
batch (i): baby mice born to mothers having received 100 tachyzoites of the Toxo mic1-3 KO strain by intraperitoneal route then infected with 15 cysts of the 76K strain of Toxoplasma gondii at D12 of gestation (black circles),
batch (ii): baby mice born to mothers not vaccinated and not infected with 15 cysts of the 76K strain of Toxoplasma gondii at D12 of gestation (black squares),
batch (iii): baby mice born to mothers having only been infected with 15 cysts of the 76K strain of Toxoplasma gondii at D12 of gestation (black triangles).

on the y-axis: cumulative clinical stage.

FIG. 5B: ophthalmological clinical signs in Swiss Webster (OF1) baby mice aged 8 weeks and infected in utero with Toxoplasma gondii.

The determination of the ophthalmological clinical signs in baby mice aged 8 weeks infected in utero with Toxoplasma gondii is carried out post-mortem using a binocular loupe. In order to evaluate the inflammation, the following scoring is used:
Stage 0: No inflammation,
Stage 1: Moderate Tyndall effect in the anterior or vitreous chamber,
Stage 2: Severe Tyndall effect in the anterior or vitreous chamber and/or dilatation of the blood vessels of the iris and/or of the conjunctiva/sclera,
Stage 3: Clouding of the cornea and retrocorneal precipitates and/or very severe hyalitis,
Stage 4: Secondary cataract.
   Each dot represents the cumulative clinical stage for a baby mouse (cumulative clinical stage being the sum of the clinical stages of the 2 eyes per baby mouse).

on the x-axis: batches of Swiss Webster (OF1) baby mice:
batch (i): baby mice born to mothers having received 100 tachyzoites of the Toxo mic1-3 KO strain by intraperitoneal route then infected with 15 cysts of the 76K strain of Toxoplasma gondii at D12 of gestation (black circles),
batch (ii): baby mice born to mothers not vaccinated and not infected with 15 cysts of the 76K strain of Toxoplasma gondii at D12 of gestation (black squares),
batch (iii): baby mice born to mothers having only been infected with 15 cysts of the 76K strain of Toxoplasma gondii at D12 of gestation (black triangles).

on the y-axis: cumulative clinical stage.

FIG. 6: evaluation of the number of intracerebral cysts in Swiss Webster (OF1) baby mice aged 8 weeks and infected in utero with Toxoplasma gondii.

Counting the number of intracerebral cysts was carried out on the basis of a brain homogenate of baby mice aged 8 weeks the mothers of which were infected or not infected at D12 of gestation with 15 cysts of the 76K strain of Toxoplasma gondii. Eight to ten counts are carried out on 10 μL of this homogenate using a binocular loupe in Malassez cells. The average is then calculated and applied to the entire initial volume in order to evaluate the number of intracerebral cysts. Each dot represents a baby mouse.

on the x-axis: batches of Swiss Webster (OF1) mice:
batch (i): baby mice born to mothers having received 100 tachyzoites of the Toxo mic1-3 KO strain by intraperitoneal route then infected with 15 cysts of the 76K strain of Toxoplasma gondii at D12 of gestation (black circles),
batch (ii): baby mice born to mothers not vaccinated and not infected with 15 cysts of the 76K strain of Toxoplasma gondii at D12 of gestation (black squares),
batch (iii): baby mice born to mothers having only been infected with 15 cysts of the 76K strain of Toxoplasma gondii at D12 of gestation (black triangles).

on the y-axis: number of intracerebral cysts.

FIG. 7: evaluation of the number of intra-retinal cysts in Swiss Webster (OF1) baby mice aged 8 weeks and infected in utero with Toxoplasma gondii.

Counting the number of intra-retinal cysts was carried out on the retina-choroid complex of the enucleated eyes of the baby mice aged 8 weeks the mothers of which were infected or not infected at D12 of gestation with 15 cysts of the 76K strain of Toxoplasma gondii. Each dot represents a baby mouse.

on the x-axis: batches of Swiss Webster (OF1) mice:
batch (i): baby mice born to mothers having received 100 tachyzoites of the Toxo mic1-3 KO strain by intraperitoneal route then infected with 15 cysts of the 76K strain of Toxoplasma gondii at D12 of gestation (black circles),
batch (ii): baby mice born to mothers not vaccinated and not infected with 15 cysts of the 76K strain of Toxoplasma gondii at D12 of gestation (black squares),
batch (iii): baby mice born to mothers having only been infected with 15 cysts of the 76K strain of Toxoplasma gondii at D12 of gestation (black triangles).

on the y-axis: number of intra-retinal cysts.

### EXAMPLE 1

### Effectiveness of the Toxo Mic1-3 KO Strain in the Prevention of Toxoplasmosis in a Murine Model of Chronic Ocular Toxoplasmosis

### 1—Experimental Protocol

1.1—Animals

Vaccination is carried out on female, non-consanguineous Swiss Webster mice (OF1) aged 8 weeks, originating from the Janvier breeding centre (Le Genest-Saint-Isle, France). Throughout the experiment the mice are kept in an animal house of containment level 2 in order to minimize the risk of external contamination.

1.2—Strain of T. gondii

1.2.1—Toxo Mic1-3 KO Strain

The mutant strain of Toxoplasma gondii, Toxo mic1-3 KO, with the genes coding for the MIC1 and MIC3 proteins knocked out, is maintained by successive passages on a human foreskin fibroblast (HFF) line cultured in DMEM medium (Dulbecco's Modified Eagle Medium) to which 10% foetal calf serum, 2 mM of L-glutamine, 100 U/mL of penicillin and 100 U/mL of streptomycin are added.

After lysis of the cell lawn, the tachyzoites are recovered from the supernatant then counted in a Malassez cell. The concentration is then adjusted in order to obtain a final dose of 100 tachyzoites in 200 μL of DMEM medium, corresponding to the vaccine dose per mouse.

1.2.2—RH Strain

The wild-type RH strain of Toxoplasma gondii, from which the Toxo mic1-3 KO strain is derived, is also maintained by successive passages on a human foreskin fibroblast (HFF) line cultured in DMEM medium to which 10% foetal calf serum, 2 mM of L-glutamine, 100 U/mL of penicillin and 100 U/mL of streptomycin are added.

For preparation of the total parasitic extract, the tachyzoites of the RH strain are washed, sonicated twice at 60 watt/s for 10 min in ice and centrifuged at 2000 g for 30 min at +4° C. The supernatant is recovered and the concentration is determined using an assay kit (BCA assay) which uses bovine serum albumin (BSA) as standard. The aliquots are stored at −20° C.

1.2.3—76K Strain

The Type II 76K strain is used for the challenge infection of the mice. This strain is preserved in the form of cysts by continuous passages in CBA/J mice by means of gavage (cycling mice). The day before the challenge infection, one cycling mouse, infected at least two months beforehand with cysts of the 76K strain, is sacrificed by cervical dislocation.

The brains of the mice are recovered after coating the fur of the animal's head with alcohol. A cutaneous incision is made using scissors, at a retroauricular nuchal line. The scalp is then anteverted and two craniotomies are carried out starting at the foramen magnum up to the frontal bones. The calvaria is then removed and the brain is taken out whole. The brain is then ground in 5 mL of RPMI medium in a Potter homogenizer. The homogenate is left overnight at +4° C.

After counting the cysts in a Malassez cell, the concentration is adjusted in order to obtain infection doses of 200 μL each containing 50 cysts.

1.3—Vaccination/Challenge Infection Protocols

Swiss-OF1 mice are divided into two separate batches:
batch (i): composed of 27 female mice vaccinated by intraperitoneal route using a 25 gauge needle. The vaccination is carried out with 100 tachyzoites of the Toxo mic1-3 KO strain, the production of which is described in paragraph 1.2.1.
batch (ii): composed of 30 unvaccinated control female mice,

Twenty-eight days after vaccination (D28), retro-orbital blood was collected from all of the mice in order to diagnose seroconversion in the mice by means of the ELISA technique.

Thirty days after vaccination (D30) the mice of batches (i) and (ii) were subjected to a challenge infection by gavage using an 18 gauge cannula, with 50 cysts of the 76K strain of Toxoplasma gondii, prepared according to the description given in paragraph 1.2.3.

Twenty-eight days after the challenge infection (D58), retro-orbital blood was taken from all of the mice.

For each batch, one-third of the animals were sacrificed 4 weeks after the gavage (D58), another third after 8 weeks (D86) and the last third after 12 weeks (D104). An ophthalmological examination is carried out on each mouse. The intracerebral and intra-retinal toxoplasmic cysts are counted and the intraocular immune response is analyzed.

1.4—Serological Analyses

The serological status of the mice is determined by an ELISA test of the indirect type.

The blood samples are centrifuged at 5,000 g for 15 min and the serum is recovered. The total parasitic extract of the RH strain, the preparation of which is described in paragraph 1.2.2, is diluted in a carbonate buffer pH 9.6 in order to obtain a final concentration of 10 μg/mL. Flat-bottom 96-well plates are then sensitized overnight at +4° C. by depositing, in each well, 100 μL of total extract of Toxoplasma gondii. The plates are then washed three times with the washing buffer (1×PBS-0.05% Tween 20) then saturated for 1 h 30 at 37° C. with a solution of 1×PBS-0.05% Tween 20 supplemented with 4% of bovine serum albumin (BSA) (Sigma). The medium is then removed.

The sera to be tested are diluted to 1/50^{th} in a solution of 1×PBS-0.05% Tween 20 and are deposited in duplicate in the wells. After incubation for one hour at 37° C. and a new series of washings, the anti-mouse IgG secondary antibody coupled with alkaline phosphatase (Sigma A3562, goat anti-Mouse IgG) and diluted to 1/5000^{th} is deposited at a rate of 100 μL per well. The samples are then incubated for one hour at 37° C. After a new series of three washings, the detection is carried out by the addition to each well of 100 μL of a solution of disodium paranitrophenylphosphate (PnPP) (Sigma) at 1 mg/mL, in a DEA-HCl buffer. After incubation for 20 min at ambient temperature and away from the light, the absorbance at 405 nm is measured by means of a plate reader (Multiskan MCC340 Wallace). The mice are considered as seroconverted when the absorbance obtained is 2.5 times greater than the absorbance obtained with the negative control originating from serum of healthy naive mice.

1.5—Ophthalmological Analyses

Before sacrificing the mice, ophthalmological analyses are carried out using a binocular loupe (Zeiss OPMI 99 colposcope floor stand to which is fixed a Zeiss F170 binocular head with its two objective lenses) after instillation of two drops of 0.1% Mydriactum in each eye with an interval of 10 min.

The fundus of the eye is then examined using this same tool with the aid of a 90-diopter “superfield” lens. The animal is brought under the light beam with a 0.6 zoom. The eye is centred, then the lens is brought to 5 mm from the eyeball without coming into contact with the latter.

1.6—Analysis of the Intraocular Immune Response

Immediately after sacrifice, aqueous humour is collected under a binocular loupe using a 30 gauge needle mounted on a 1 mL syringe. The two samples corresponding to the two eyes of the same mouse are combined and the intraocular IFN-γ is assayed by means of ELISA (BD Opt EIA Mouse IFN-γ ELISA set). On D1, the capture antibody diluted 1/250 in “coating” buffer (dilution buffer: 85 mM of NaHCO₃, 15 mM of Na₂CO₃, pH9.5) is deposited on 96-well plates. These plates are incubated at +4° C. overnight. After washing with 1×PBS-0.05% Tween 20 buffer, the plate is saturated for 1 h at ambient temperature with saturation buffer (1×PBS-10% FCS) at a rate of 200 μL per well. After a new series of three washings, the samples of aqueous humour diluted to 1/10^{th} in saturation buffer are deposited then incubated for two hours at ambient temperature at a rate of 50 μL per well. In parallel, a range is produced based on commercially obtained murine IFN-γ. After a new series of washings, 50 μL of the solution containing the antibody and the detection enzyme are deposited diluted to 1/250^{th} in saturation buffer and incubated for 1 h. After a new series of washings, the plate is developed with the substrate (Tetramethylbenzidine, Sigma), at a rate of 50 μL per well. After 30 min, 25 μL of stop solution (2N H₂S0₄) is added. The optical densities are read at 450 nm using a plate reader (Multiskan MCC340 Wallace).

1.7—Counting the Intraretinal and Intracerebral Cysts

1.7.1—Intracerebral Cysts

The brains of the mice are recovered after coating the fur of the animal's head with alcohol. A cutaneous incision is made with scissors, at a retroauricular nuchal line. The scalp is then anteverted and two craniotomies are carried out starting at the foramen magnum up to the frontal bones. The calvaria is then removed and the brain is taken out whole. The brain is then ground in 5 mL of RPMI medium in a Potter homogenizer. The homogenate is left overnight at +4° C.

For counting the cysts, eight to ten counts are carried out on 10 μL of this homogenate using a binocular microscope in Malassez cells. The average is then calculated and applied to the total initial volume in order to assess the number of intracerebral cysts.

1.7.2—Intraretinal Cysts

After removal of the aqueous humour, the eyes of the mice are enucleated. Under a binocular loupe, a total conjunctival debridement is performed. A limbic incision is made and the cornea is extracted. Four orthogonal scleral incisions are then made towards the posterior pole using Vannas scissors and the retina-choroid-sclera complex is spread out flat on the work surface. The crystalline lens is also removed, keeping as much of the vitreous gel as possible in contact with the retina in order to minimize trauma. The retina-choroid complex is then carefully removed using a micromanipulator and placed in 50 μL of RPMI medium, then homogenized by moving back and forth in the tip of a micropipette.

For counting the cysts, the whole of the sample is observed between slide and coverslip using a binocular loupe.

### 2—Results

2.1.—Experimental Procedure

Thirty days after vaccination, the mice were subjected to a challenge infection by gavage with 50 cysts of the 76K strain.

2.2.—Serological Analyses

One month after the vaccination, a serological analysis was carried out on all the animals. The 27 vaccinated animals of batch (i) have an antibody titre greater than 2.5 times that of the control mice of batch (ii) and are therefore considered as seroconverted vis-à-vis Toxoplasma gondii. By contrast, the mice of the control batch remain seronegative.

A second serological analysis was carried out, one month after the challenge infection. After the challenge infection, all of the mice of batch (i) which are vaccinated and infected, and of batch (ii) which are only infected are seropositive. Moreover, the optical density observed based on the sera of the vaccinated and infected mice is greater than that observed based on the mice that are only vaccinated or only infected, thus reflecting a higher level of antibodies in the vaccinated and infected mice compared with that observed in the mice that are only vaccinated or only infected.

2.3.—Ophthalmological Analyses

Four weeks post-challenge infection, thirty-eight eyes were examined (18 in the batch that were vaccinated then infected—batch (i), and 20 in the infected control batch—batch (ii). The results are shown in FIG. 1A. In the infected control batch (batch (ii)), eight eyes showed signs of inflammation: 7 were of the uveitis type (35%) and only one of the haemorrhagic type (5%). These signs were therefore found in 40% of the eyes analyzed. In the vaccinated then infected batch (batch (i)), no inflammatory lesion was observed, but one animal had bilateral corneal opacity (11% of the eyes analyzed) and another had cataracts (11% of the eyes analyzed).

At eight weeks post-challenge infection, thirty-eight eyes were examined (18 in the vaccinated then infected batch—batch (i), and 20 in the infected control batch—batch (ii)). In the infected control batch (batch (ii)), six eyes showed signs of inflammation of the uveitis type (30%), two eyes showed signs of cataracts (10%) and only one showed signs of the haemorrhagic type (5%). These signs were therefore found in 45% of the eyes analyzed. In the vaccinated then infected batch (batch (i)), no inflammatory lesion was observed but one animal, again, had bilateral corneal opacity (FIG. 1B).

At twelve weeks post-challenge infection, thirty-six eyes were examined (16 in the vaccinated then infected batch—batch (i) and, 20 in the control batch—batch (ii)). In the infected control batch (batch (ii)), five eyes showed signs of inflammation of the uveitis type (25%) and three of cataract (15%). These signs were therefore found in 40% of the eyes analyzed. In the vaccinated then infected batch (batch (i)), no inflammatory lesion was observed (FIG. 1C).

In summary, lesions were found in 41.6% of the eyes of the unvaccinated and infected control mice (batch (ii)). The main clinical signs observed are of hyalitis (30%), haemorrhages (3.3%) and cataracts (8.3%). In the vaccinated and infected batch (batch (i)), only 7.7% of the eyes had bilateral corneal lesions. Furthermore, as these clinical signs are not described as a consequence of a T. gondii infection, they are very probably due to another cause such as trauma suffered in the cages or during handling.

2.4. —Counting the Intracerebral and Intraretinal Cysts

2.4.1—Intracerebral Cysts

The presence of intracerebral cysts was sought in the brains of the control mice that were only infected (batch (ii)), and vaccinated and infected (batch (i)):
At four weeks post-challenge infection, the control mice (batch (ii)) had an average number of intracerebral cysts of 718±187 cysts whereas no cysts were counted in the brains of the mice vaccinated beforehand (batch (i)) (FIG. 2A),
At eight weeks post-challenge infection, the control mice (batch (ii)) had an average number of intracerebral cysts of 880±394 cysts whereas no cysts were counted in the brains of the mice vaccinated beforehand (batch (i)) (FIG. 2B),
At twelve weeks post-challenge infection, the control mice (batch (ii)) had an average number of intracerebral cysts of 1,094±303 cysts whereas no cysts were counted in the brains of the mice vaccinated beforehand (batch (i)) (FIG. 2C).

2.4.2—Intraretinal Cysts

The presence of intraretinal cysts was sought in the eyes of the control mice that were only infected (batch (ii)), and vaccinated and infected (batch (i)):
At four weeks post-challenge infection, the control mice (batch (ii)) had an average number of cysts per eye of 4.50±2.26 as against 0.66±0.77 cysts per eye in the case of the mice of the vaccinated batch (batch (i)) (FIG. 3A),
At eight weeks post-challenge infection, the control mice (batch (ii)) had an average number of cysts per eye of 5.55±3.56 as against 0.44±0.51 cysts per eye in the case of the mice of the vaccinated batch (batch (i)) (FIG. 3B),
Finally, at twelve weeks post-challenge infection, the control mice (batch (ii)) had an average number of cysts per eye of 4.3±2.66 as against 0.31±0.60 cysts per eye in the case of the mice of the vaccinated batch (batch (ii)) (FIG. 3C).

This experiment made it possible to demonstrate a clear reduction in the number of intraretinal cysts in the vaccinated mice (batch (i)) in comparison with the unvaccinated control mice (batch (ii)). Thus, only 40% of the eyes (21/52 eyes) of the vaccinated mice (batch (i)) had intraretinal cysts as against 96% of the eyes of the control mice (batch (ii)) (58/60 eyes). Similarly, the average number of intraretinal cysts is 4.8±2.85 cysts per eye in the case of the control mice (batch (ii)) as against 0.46±0.63 cysts per eye in the case of the vaccinated mice (batch (i)), i.e. a greater than 90% reduction.

2.5.—Analysis of the Immune Response

Samples of aqueous humour were taken from the mice of the control batch (batch (ii)) and the vaccinated batch (batch (i)). The cytokine IFN-γ in the aqueous humour was assayed. The ocular defence is produced by increasing the production of IL-12 which in turn induces secretion of IFN-γ and TNF-α. This inflammatory intraocular environment is highly deleterious to the eye.

Four weeks after the challenge infection, the average intraocular secretion of IFN-γ in the unvaccinated but infected control individuals (batch (ii)) is 7,659±7,980 pg/mL (FIG. 4A). Still in the animals of batch (ii), this intraocular secretion is 722±1,045 pg/mL at eight weeks post-infection (FIG. 4B) and 1,200±1,866 pg/mL at twelve weeks post-infection (FIG. 4C). This reduction is explained by the spontaneous development of inflammatory flare-ups, which resolve in approximately 4 weeks.

In the case of the mice vaccinated beforehand with the Toxo mic1-3 KO strain (batch (i)), no local secretion of IFN-γ is detected, four (34±42 pg/ml), eight (21±34 pg/mL) or twelve (1±2 pg/mL) weeks after the infection (FIGS. 4A, 4B and 4C), indirectly indicating the absence of inflammatory processes that are deleterious to the eyes.

### EXAMPLE 2

### Effectiveness of the Toxo Mic1-3 KO Strain for the Prevention of Toxoplasmosis in a Murine Model of Congenital Ocular Toxoplasmosis

1—Experimental Protocol

1.1—Animals

The vaccination is carried out on female, non-consanguineous Swiss Webster mice (OF1), aged 8 weeks, originating from the Janvier breeding centre (Le Genest-Saint-Isle, France). Throughout the experiment the mice are kept in an animal house of containment level 2 in order to minimize the risk of external contamination.

1.2—T. gondii Strains

1.2.1—Toxo Mic1-3 KO Strain

The mutant strain of Toxoplasma gondii, Toxo mic1-3 KO, with the genes coding for the proteins MIC1 and MIC3 knocked out, is maintained by successive passages on a human foreskin fibroblast (HFF) line cultured in DMEM medium (Dulbecco's Modified Eagle Medium) to which 10% foetal calf serum, 2 mM of L-glutamine, 100 U/mL of penicillin and 100 U/mL of streptomycin are added.

After lysis of the cell lawn, the tachyzoites are recovered from the supernatant then counted in a Malassez cell. The concentration is then adjusted in order to obtain a final dose of 100 tachyzoites in 200 μL of DMEM medium, corresponding to the vaccine dose per mouse.

1.2.2—RH Strain

The wild-type RH strain of Toxoplasma gondii, from which the Toxo mic1-3 KO strain is derived, is also maintained by successive passages on a human foreskin fibroblast (HFF) line cultured in DMEM medium to which 10% foetal calf serum, 2 mM of L-glutamine, 100 U/mL of penicillin and 100 U/mL of streptomycin are added.

For preparation of the total parasitic extract, the tachyzoites of the RH strain are washed, sonicated twice at 60 watts for 10 min in ice and centrifuged at 2,000 g for 30 min at +4° C. The supernatant is recovered and the concentration is determined using an assay kit (BCA assay) which uses bovine serum albumin (BSA) as standard. The aliquots are stored at −20° C.

1.2.3—76K Strain

The Type II 76K strain is used for the challenge infection of the mice. This strain is preserved in the form of cysts by continuous passages in CBA/J mice by gavage (cycling mice). The day before the challenge infection, one cycling mouse, infected two months beforehand with cysts of the 76K strain, is sacrificed by cervical dislocation.

The brains of the mice are recovered after coating the fur of the animal's head with alcohol. A cutaneous incision is made with scissors, at a retroauricular nuchal line. The scalp is then anteverted and two craniotomies are carried out starting at the foramen magnum up to the frontal bones. The calvaria is then removed and the brain is taken out whole. The brain is then ground in 5 mL of RPMI medium in a Potter homogenizer. The homogenate is left overnight at +4° C.

The cysts are then counted in a Malassez cell and the concentration is adjusted in order to obtain infection doses of 200 μL each containing 15 cysts.

1.3—Vaccination/Challenge Infection Protocols

Female Swiss-OF1 mice are divided into three separate batches:
batch (i) constituted by 10 vaccinated and infected female mice (vaccinated/infected batch). The mice are vaccinated on the first day of the experiment (D0) by intraperitoneal route using a 25 gauge needle. The vaccination is carried out with 100 tachyzoites of the Toxo mic1-3 KO strain, the production of which is described in paragraph 1.2.1.,
batch (ii) constituted by 5 unvaccinated and uninfected female mice (unvaccinated and uninfected batch),
batch (iii) constituted by 15 unvaccinated and infected female mice (unvaccinated and infected batch).

Twenty-eight days after vaccination (D28), submaxillary blood was taken from all of the vaccinated mice in order to diagnose the seroconverted mice (vaccinated/infected batch—batch (i)).

Two months after vaccination, the seropositive mice of the vaccinated/infected batch (batch (i)) as well as all the mice of the unvaccinated/uninfected batch (batch (ii)) and of the unvaccinated/infected batch (batch (iii)) were put to the male for 3 days at a rate of 3 female mice per male. The pregnant mice were diagnosed by weighing.

On the twelfth day of gestation, the diagnosed pregnant mice of the vaccinated/infected batch (batch (i)) and of the unvaccinated and infected batch (batch (iii)) were subjected to a challenge infection by gavage using an 18 gauge cannula, with 15 cysts of the 76K strain, the preparation of which is described in paragraph 1.2.3.

Four weeks after birth, the young mice undergo an ophthalmological examination carried out under general anaesthesia obtained by inhalation of 2.5% gaseous isoflurane (oxygen at 31 per minute) in an induction cage unit (Mark 5, Minerve SA) for 2 to 5 minutes, per batch of 5 mice maximum.

Eight weeks after birth, the young mice are sacrificed and a second ophthalmological examination is carried out. The ocular and cerebral cysts are counted and the intraocular immune response is analyzed.

1.4—Serological Analysis

The serological status of the mice is determined by means of an ELISA test of the indirect type. In the case of the mothers, the serological status was studied 4 weeks after vaccination and 4 and 8 weeks after the challenge infection. In the case of the young mice, the serological status was studied at the fourth and eighth week of life.

The blood samples are centrifuged at 5,000 g for 15 min and the serum is recovered. The total extract of the RH strain the preparation of which is described in paragraph 1.2.2 is diluted in a carbonate buffer pH 9.6 in order to obtain a final concentration of 10 μg/mL. Flat-bottom 96-well plates are then sensitized overnight at +4° C. by depositing 100 μL of total extract of Toxoplasma gondii in each well. The plates are then washed three times with the washing buffer (1×PBS-0.05% Tween 20) then saturated for 1 h 30 at 37° C. with a solution of 1×PBS-0.05% Tween 20 supplemented with 4% of bovine serum albumin (BSA) (Sigma). The medium is then removed.

The sera to be tested are diluted to 1/50^{th} in a solution of 1×PBS-0.05% Tween 20 and deposited in duplicate in the wells. After incubation for one hour at 37° C. and a new series of washings, the anti-mouse IgG secondary antibody coupled with alkaline phosphatase (Sigma A3562, goat anti-Mouse IgG) and diluted to 1/5000^{th} is deposited at a rate of 100 μL per well. The samples are then incubated for one hour at 37° C. After a new series of three washings, detection is carried out by the addition to each well of 100 μL of a solution of disodium paranitrophenylphosphate (PnPP) (Sigma) at 1 mg/mL, in a DEA-HCl buffer. After incubation for 20 min at ambient temperature and away from the light, the absorbance at 405 nm is measured by means of a plate reader (Multiskan MCC340 Wallace). The mice are considered as seroconverted when the absorbance obtained is 2.5 times greater than the absorbance obtained with the negative control originating from serum of healthy, naive mice.

1.5—Ophthalmological Analyses

For each young mouse, we were able to carry out ophthalmological examinations. The first is carried out after 4 weeks of life on anaesthetized individuals according to the protocol previously described. The second is carried out at 8 weeks of life, after euthanasia.

After anaesthesia or euthanasia of the mice, ophthalmological analyses are carried out using a binocular loupe (Zeiss OPMI 99 colposcope floor stand to which is fixed a Zeiss F170 binocular head with its two objective lenses) after instillation of two drops of 0.1% Mydriactum® in each eye.

The anterior segment is examined, ensuring corneal hydration in order to prevent the onset of exposure keratitis or corneal oedema capable of creating artefacts. The ophthalmological examination made it possible to note the occurrence of anterior uveitis (pigmented retro-corneal precipitates) and/or cataract (crystalline opalescence, sutural or subcapsular cataracts or even true nuclear cataract), hyalitis or retinal haemorrhage.

In order to assess the inflammation, the following scoring was used (Sauer et al., 2009 Journal Français d' Ophthalmologie, 32: 742-749):
Stage 0: no inflammation,
Stage 1: Moderate Tyndall effect in the anterior or vitreous chamber,
Stage 2: Severe Tyndall effect in the anterior or vitreous chamber and/or dilatation of the blood vessels of the iris and/or of the conjunctiva/sclera,
Stage 3: Clouding of the cornea and retrocorneal precipitates and/or very severe hyalitis,
Stage 4: Secondary cataract.

1.6—Counting the Intraretinal and Intracerebral Cysts

1.6.1—Intracerebral Cysts

The brains of the mice are recovered after coating the fur of the animal's head with alcohol. A cutaneous incision is made with scissors, at a retroauricular nuchal line. The scalp is then anteverted and two craniotomies are carried out starting at the foramen magnum up to the frontal bones. The calvaria is then removed and the brain is taken out whole. The brain is then ground in 5 mL of RPMI medium in a Potter homogenizer. The homogenate is left overnight at +4° C.

For the cyst count, eight to ten counts are carried out on 10 μL of this homogenate using a binocular loupe in Malassez cells. The average is then calculated and applied to the total initial volume in order to assess the number of intracerebral cysts.

1.6.2—Intraretinal Cysts

After removal of the aqueous humour, the mice are enucleated. Under a binocular loupe, a total conjunctival debridement is performed. A limbic incision is made and the cornea is removed. Four orthogonal scleral incisions are then made towards the posterior pole using Vannas scissors, and the retina-choroid-sclera complex is spread out flat on the work surface. The crystalline lens is also removed, keeping as much of the vitreous gel as possible in contact with the retina in order to minimize trauma. The retina-choroid complex is then carefully removed using a micromanipulator and placed in 50 μL of RPMI medium, then homogenized by moving back and forth in the tip of a micropipette.

For counting the cysts, the whole of the sample is observed between slide and coverslip, using a binocular loupe.

1.7—Analysis of the Intraocular Immune Response

Immediately after sacrifice, aqueous humour is collected under a binocular loupe using a 30 gauge needle mounted on a 1 mL syringe. The two samples corresponding to the two eyes of the same mouse are combined and the intraocular IFN-γ is assayed by means of ELISA (BD Opt EIA Mouse IFN-γ ELISA set). At D1, the capture antibody diluted to 1/250^{th} in “coating” buffer (dilution buffer: 85 min of NaHCO₃, 15 min of Na₂CO₃, pH 9.5) is deposited on 96-well plates. These plates are incubated at +4° C. overnight. After washing with 1×PBS-0.05% Tween 20 buffer, the plate is saturated for 1 h at ambient temperature with saturation buffer (1×PBS, 10% FCS) at a rate of 200 μL per well. After a new series of three washings, the samples of aqueous humour diluted to 1/10^{th} in saturation buffer are deposited then incubated for two hours at ambient temperature (50 μL per well for the IFN-γ). In parallel, a range is produced based on commercially obtained murine IFN-γ. After a new series of washings, 50 μL of the solution containing the antibody and the detection enzyme are deposited diluted to 1/250^{th} in saturation buffer and incubated for 1 h. After a new series of washings, the plate is developed with the substrate (Tetramethylbenzidine), at a rate of 50 μL per well. After 30 min, 25 μL of stop solution (2N H₂SO₄) is added. The optical densities are read at 450 nm using a plate reader (Multiskan MCC340 Wallace).

### 2—Results

2.1.—Experimental Procedure

With the aim of demonstrating the effectiveness of the Toxo mic1-3 KO strain in the prevention of ocular toxoplasmosis by mother-fœtus transmission, female mice were vaccinated, put to the male and infected at mid-gestation with a wild-type strain of T. gondii.

The mice of batch (i), as well as the control mice of batches (ii) and (iii) were put to the male. Then, the mice of batches (i) and (iii) were subjected to a challenge infection by oral route on the twelfth day of gestation, with 15 cysts of the 76K strain of T. gondii.

The perinatal mortality was assessed during the first 4 weeks of life. Thus, during this period, 6 young mice out of 127 died in the vaccinated batch (i) i.e. 4.72%.

In batch (ii), constituted by unvaccinated and uninfected control mice, only one young mouse out of 43 died (2.3%) and, in batch (iii) constituted by unvaccinated and infected control mice, 27 young mice out of 83 died (32.5%).

2.2.—Ophthalmological Analyses

The clinical examinations carried out at the fourth week of life of the young mice were carried out under general anaesthesia in the 219 young mice (121 for batch (i), 42 for batch (ii) and 56 for batch (iii)).

In batch (i), constituted by young mice originating from vaccinated and infected mothers, 13.1% of the young mice showed clinical signs as against 71.4% in batch (iii), constituted by the young mice originating from unvaccinated but infected mothers (p<0.0001). In batch (ii), constituted by young mice originating from unvaccinated and uninfected mothers, only 3 young mice showed clinical signs of intraocular inflammation i.e. 7.1%, with no significant difference compared with batch (i) (p=0.52).

The average cumulative clinical stage (FIG. 5A) (the sum of the clinical stages of both eyes of each young mouse) is significantly lower in batch (i) (0.23±0.64) than in batch (iii) (1.59±1.83) (p<0.0001). On the other hand, the average cumulative clinical stage is comparable in batch (i) and in batch (ii) (0.15±0.65) (p=0.52).

A new ophthalmological examination was carried out on these same young mice sacrificed at the age of eight weeks.

In batch (i), only 23 young mice out of 121 showed clinical signs of intraocular inflammation, i.e. 19%. By contrast, in batch (iii), 41 young mice out of 56 had symptomatic inflammation, i.e. 73.2% (p<0.0001). The average cumulative clinical stage of the young mice of batch (i) is also significantly lower (0.85±1.92) than that of the young mice of batch (iii) (4.37±3.10) (FIG. 5B).

On the other hand, no significant difference is noted between batches (i) and (ii), both with regard to the number of young mice with symptomatic inflammations (3 out of 42 young mice, i.e. 7.1% of the young mice of batch (ii), i.e. p=0.0711), and with regard to the level of the average cumulative clinical stage (0.32±1.24 for the young mice of batch (ii), i.e. p=0.094).

On the other hand, an increase is noted in the cumulative clinical stages per young mouse over time, between the fourth and eighth week of life (FIGS. 5A and 5B).

2.3.—Counting the Intracerebral and Intraretinal Cysts

2.3.1—Intracerebral Cysts

A sample of brain was taken from 121 young mice of batch (i), 36 young mice of batch (ii) (6 young mice not sampled) and 50 young mice of batch (iii) (6 not sampled). A significant difference is observed between the number of young mice with a cerebral infection in batch (i) (36.4%) and the number of young mice with a cerebral infection in batch (iii) (98%) (p<0.0001). No cerebral cysts were detected in the young mice of batch (ii).

The average number of cysts per brain (FIG. 6) is also very significantly lower in the young mice of batch (i) (61±159) compared with the young mice of batch (iii) (282±190) (p<0.0001).

2.3.2—Intraretinal Cysts

All the eyes of the 219 young mice were removed (121 in the case of batch (i), 42 in the case of batch (ii) and 56 in the case of batch (iii)).

A young mouse is considered to be ocularly infected when at least one cyst is observed in the retina of at least one of its two eyes. The level of ocular infection is very significantly lower in the young mice of batch (i) (24% of the ocularly infected young mice) than in the young mice of batch (iii) (71.4% of the ocularly infected young mice). No young mouse of batch (ii) had any intraretinal cysts.

Furthermore, the average number of intraretinal cysts per young mouse (FIG. 7) is also very significantly lower in the animals of batch (i) (0.56±1.18 cysts by eye) than in the animals of batch (iii) (2.54±4.10 cysts per eye).

2.4—Analysis of the Serological Response

At the fourth week of life, the ELISA test for assay of the serum anti-T. gondii antibodies, performed according to the procedure described in paragraph 1.4, shows that 98% of the young mice of batch (i) were seropositive as against 34% of the young mice of batch (iii). No young mouse of batch (ii) had any anti-T. gondii antibodies.

Thus, the optical density observed at 405 nm was significantly lower in the sera of the young mice of batch (iii) than in the sera of the young mice of batch (i) (p=0.008), reflecting a lower level of antibodies in the young mice of batch (iii) than in the young mice of batch (i).

At the eighth week of life, no additional seroconversion was found in the young mice of batch (i), 98% of young mice having serum antibodies. On the other hand, 100% of the young mice of batch (iii) were seropositive. All of the young mice of batch (ii) were seronegative.

2.5—Analysis of the Intraocular Immune Response

It was possible to test all of the eyes of the young mice.

The average concentration of IFN-γ in the anterior chamber was 1.170.5±1.918.7 pg/mL in the young mice of batch (i), 2.147.6±3.917.5 pg/mL in the young mice of batch (iii) and 940.9±180.0 pg/mL in the young mice of batch (ii).

The average concentration of IFN-γ in the anterior chamber is significantly reduced in the young mice of batch (i) compared to that observed in the young mice of batch (iii) (p=0.027), but, for this parameter, there is no statistically significant difference between the young mice of batch (i), on the one hand, and those of batch (ii), on the other hand (p=0.44).

Furthermore, the concentration of IFN-γ in the anterior chamber is correlated with the average number of retinal cysts (p<0.05, R²=0.144) as well as with the average cumulative clinical stages observed at the fourth and eighth weeks of life (p<0.05, R²=0.186 and R²=0.224 respectively).

Finally the average cumulative clinical stage at the eighth week of life is strictly correlated with the average number of intraretinal cysts (p<0.05, R²=0.542).

1. Strains of Toxoplasma gondii isolated from their natural environment for their use in the prevention and/or the treatment, in a mammal, of ocular lesions associated with an infection by an apicomplexan of the Sarcocystidae family.

2. Strains of Toxoplasma gondii isolated from their natural environment for their use according to claim 1, said strains having an attenuated virulence in comparison with a virulent strain of T. gondii of RH type.

3. Strains of Toxoplasma gondii isolated from their natural environment for their use according to claim 1, in which said mammal is a human being or an animal.

4. Strains of Toxoplasma gondii isolated from their natural environment for their use according to claim 1, in which said strains of Toxoplasma gondii have at least one adhesin MIC-1 and/or one adhesin MIC-3 inactivated by a genetic modification involving at least one of the mic-1 and/or mic-3 genes.

5. Strains of Toxoplasma gondii isolated from their natural environment for their use according to claim 1, in which said strains of Toxoplasma gondii have the two adhesins MIC-1 and MIC-3 inactivated by a genetic modification involving the two mic-1 and mic-3 genes.

6. Strains of Toxoplasma gondii isolated from their natural environment for their use according to claim 1, in which said apicomplexan of the Sarcocystidae family is Toxoplasma gondii.

7. Strains of Toxoplasma gondii isolated from their natural environment for their use according to claim 1, in which said ocular lesions belong to the group comprising or constituted by intraocular inflammations, uveitis, hyalitis or retinochoroiditis.

8. Strains of Toxoplasma gondii isolated from their natural environment for their use according to claim 1, in which said strains are placed in contact with said mammal at a rate of 100 to 10⁸ tachyzoites.

9. Strains of Toxoplasma gondii isolated from their natural environment for their use according to claim 1, in which said strains are in a galenic form selected from the group comprising or constituted by liquid suspensions, solid or liquid dispersions, powders, pastes or lyophilizates.

10. Strains of Toxoplasma gondii isolated from their natural environment for their use according to claim 1, in which said strains are combined with at least one other antigen, at least one adjuvant, at least one stabilizer, at least one preservative or a mixture of said products making it possible to stimulate and increase the immune response of said mammal.
